(19) United States
(12) Patent Application Publication
(10) Pub. No.: US 2005/0175559 A1
(43) Pub. Date: Aug. 11, 2005

(54) Method and preparation for reducing skin hyperpigmentation

(75) Inventors: Joseph C. DiNardo, Raleigh, NC (US); Joseph A. Lewis II, Chesterfield, VA (US); Birgit Neudecker, Munich (DE); Falko Diedrich, Munich (DE); Eberhard Wieland, Stuttgart (DE)

Correspondence Address: DAVIDSON, DAVIDSON & KAPPEL, LLC, 485 SEVENTH AVENUE, 14TH FLOOR, NEW YORK, NY, 10018, US

(73) Assignee: PCR Technology Holdings, LC, Richmond, VA (US)

(21) Appl. No.: 11/010,854

(22) Filed: Dec. 13, 2004

Related U.S. Application Data
(60) Provisional application No. 60/543,435, filed on Feb. 10, 2004.

(51) Int. Cl. A61K 7/135; A61K 31/445

(52) U.S. Cl. 424/62; 514/327

(57) ABSTRACT

A method for reducing an occurrence of hyperpigmentation in human skin includes applying to the skin a topical preparation including an amount of an agent effective to reduce an occurrence of hyperpigmentation in human skin. The agent includes idebenone or a derivative of idebenone.

Priority is claimed to provisional application 60/543,435, filed Feb. 10, 2004.

The present invention relates in general to human skin, and in particular to reduction of hyperpigmentation in human skin.

### BACKGROUND

Hyperpigmentation is generally related to dense melanin concentrations in skin tissues. The color of skin is determined by a composite of red (oxyhemoglobin), blue (reduced hemoglobin), yellow (carotinoids and flavins), and brown (melanin). Of these various pigments, melanin content is the most variable, and has become synonymous with skin pigmentation. Hyperpigmentation of the skin occurs when there is an increased production of melanin and results in localized areas of increase skin pigmentation. Spots of hyperpigmentation may result from natural phenomena or they may be caused by external stress, drugs, or other dysfunctions. Exposure to ultraviolet (UV) radiation is known to provide a variety of undesirable effects, including hyperpigmentation. Most common types of localized hyperpigmentation includes: lentigo (sun spots, liver spots), ephelis (freckles), nevus (moles), increased pigment due to seborrheic or actinic keratoses and melasma (mask of pregnancy). The last type corresponds to hyperpigmentation in pregnant women or women taking oral contraceptives and is due to increased level of melanocyte-stimulating hormone and adrenocorticotrophic hormone produced by the pituitary gland. Exposure to inflammatory skin reactions may cause a type of reaction knows as PIH (Post Inflammatory Hyperpigmentation). PIH is a result from over exposure to topical application of chemicals, of cosmetics and/or drugs that have a potential to evoke an erythema/edema response in skin. Thus, events known or suspected to cause hyperpigmentation include an exposure to ultraviolet radiation, an exposure to a chemical, an exposure to a cosmetic and an exposure to a drug.

To enhance even color tone of the skin, physicians and skincare professionals have used hydroquinone. Hydroquinone lightens areas of hyperpigmentation.

Traditional sunscreen compositions containing either physical or chemical sunscreens do provide some degree of protection against hyperpigmentation. However, these products still permit significant hyperpigmentation in skin which is exposed to UV radiation.

U.S. Pat. No. 4,593,038 describes a method of cosmetically treating skin to reduce hyperpigmentation by topically applying to affected skin areas 3-phenylacetylamino-2,6-piperidinedione dispersed in a suitable cosmetic vehicle.

### SUMMARY OF THE INVENTION

The present invention provides a method for reducing an occurrence of hyperpigmentation in human skin. The occurrence of hyperpigmentation may be due to a variety of causes, including an exposure to ultraviolet radiation, a chemical, a cosmetic and/or a drug, as well as to disease and/or an inflammatory skin response. The method includes applying to the skin a topical preparation including an amount of an agent effective to reduce an occurrence of hyperpigmentation in human skin. The agent includes idebenone or a derivative of idebenone.

The idebenone or derivative of idebenone may have a concentration of from about 0.001% to about 30%, by weight of the preparation. The preparation may be in the form of a lotion, a cream, a gel, a solution, a spray, a cleanser, a powder, an ointment, a wax, a lipstick, a soap, a shampoo, a hydroalcoholic solution, a suspension, a scrub, a saturated pad, or a skin or hair conditioning agent.

The present invention also provides a method for reducing an occurrence of hyperpigmentation in human skin, the method including: providing a person in need of a reduction of an occurrence of hyperpigmentation, the hyperpigmentation being responsive to an application of idebenone or a derivative of idebenone; and applying to the skin a topical preparation including an amount of an agent effective to reduce the occurrence of hyperpigmentation in human skin. The agent includes idebenone or a derivative of idebenone.

Also provided by the present invention is a method for reducing an occurrence of hyperpigmentation in human skin, the method including making available a topical preparation directed to reducing an occurrence of hyperpigmentation in human skin, and providing an information on applying the topical preparation to the skin. The topical preparation includes an amount of an agent effective to reduce the occurrence of hyperpigmentation in human skin. The agent includes idebenone or a derivative of idebenone.

A topical preparation for reducing an occurrence of hyperpigmentation in human skin is also provided by the present invention. The topical preparation includes at least one of an ultraviolet filter substance, a depigmentation substance and an antioxidant, and an amount of idebenone or a derivative of idebenone effective to reduce an occurrence of hyperpigmentation in human skin.

According to an embodiment the applying of the topical preparation may be performed after the hyperpigmentation has occurred. In another embodiment, the applying is performed prior to an event known or suspected to cause hyperpigmentation.

It has been found in clinical testing that the method and preparation of the invention provided, for example, a 50% reduction in hyperpigmentation by treating human skin with a idebenone-containing compositions (0.5 wt. % and 1.0 wt. %).

### DETAILED DESCRIPTION

Idebenone (6-(10-hydroxydecyl)-2,3-dimethoxy-5-methyl-1,4-benzoquinone) is characterized by the following structural formula:

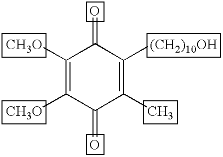

Chemical derivatives of idebenone may also be suitable for use in a method according to the present invention. Such derivatives may include, for example, esters and salts of idebenone, or protein bound forms, or other derivatives. Examples of idebenone derivatives include esters of idebenone where idebenone is esterified using glycosaminoglycans and/or their salts, for example hyaluronic acid having a molecular weight of 1 to 1,000,000 and its salts or hyaluronidase inhibitors, such as for example inter-alpha-trypsin inhibitor.

An example of a hydrophilic idebenone ester (separate synthesis) is idebenone sulphonic acid, characterized by the following structural formula:

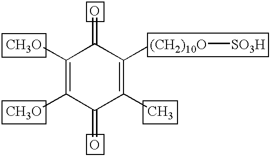

An exemplary synthesis of idebenone sulphonic acid was performed as follows: idebenone was reacted with pyridine-SO₃ and the reaction was then stopped using 1 N hydrochloric acid. After shaking out the organic phase using ethyl acetate, the organic phase was dried and concentrated under vacuum. The residue was dissolved in water and insoluble products centrifuged off. The hydrophilic idebenone ester thus recovered is suitable for application according to the invention in aqueous cosmetic and dermatological preparations.

Compositions according to the present invention may contain a concentration of idebenone or a derivative of idebenone of about 0.001-30%, 0.01-10.0%, 0.1-2.0%, or 0.5% to 1.0% by weight of the composition.

The compositions may be cosmetic, dermatologic, or pharmaceutical preparations or compositions, and may exist in a wide variety of forms, such as emulsions, suspensions, solutions and the like. In certain embodiments, the compositions are in the form of lotions, creams, gels, solutions, sprays, cleansers, powders, ointments, waxes, lipsticks, soaps, shampoos, hydroalcoholic solutions, suspensions, scrubs, saturated pads, skin or hair conditioning agents, and other types of cosmetic compositions.

For administration, the cosmetic and dermatological preparations of the invention may be applied to the skin in adequate quantity in the manner conventional for cosmetics.

Cosmetic and dermatological preparations of the invention may exist in various forms. Hence, they may be, for example a solution, an anhydrous preparation, an oil-free preparation, an emulsion or microemulsion of the type water-in-oil (W/O) or of the type oil-in-water (O/W), a multiple emulsion, for example of the type water-in-oil-in-water (W/O/W), a gel, a solid stick, an ointment or even an aerosol. It is also advantageous to administer idebenone and/or its derivatives in encapsulated form, for example in collagen matrices and other conventional encapsulation materials, for example as cellulose encapsulations, in gelatine, wax matrices or liposomally encapsulated.

It is also possible and advantageous within the scope of the present invention to add idebenone and/or its derivatives, such the sulphate of idebenone, for example, to aqueous systems or surfactant preparations for cleansing the skin.

The cosmetic and dermatological preparations of the invention may contain cosmetic auxiliaries, as are used conventionally in such preparations, for example preservatives, bactericides, perfumes, substances for preventing foaming, dyestuffs, pigments which have a coloring effect, thickening agents, surfactant substances, emulsifiers, softening, moisturizing and/or moisture-retaining substances, fats, oils, waxes or other conventional constituents of a cosmetic or dermatological formulation, such as alcohols, polyols, polymers, foam stabilizers, electrolytes, organic solvents or silicone derivatives.

In particular, idebenone and its derivatives may also be combined according to the invention with one or more traditional or other anti-oxidants and/or free-radical absorbers that are suitable or conventional for cosmetic and/or dermatological applications. Such anti-oxidants include, for example, one or more of the following: antioxidant enzymes (for example superoxide dismutase, catalase, glutathione peroxidase, glutathione S-transferase, glutathione reductase), antioxidant polyphenols such as those present in many botanical extracts (for example, green tea, white tea, black tea, licorice, grape, bilberry, mulberry, pomegranate, soy) including but not limited to flavones, isoflavones, cyanidins, anthocyanidins, catechins and related compounds (for example, chlorogenic acid, caffeic acid, ellagic acid, genistein), plant growth factors (for example N-furfuryladenine), amino acids (for example glycine, histidine, tyrosine, tryptophan) and their derivatives, imidazoles (for example urocanic acid) and their derivatives, peptides, such as D,L-camosine, D-carnosine, L-carnosine and their derivatives (for example anserine), carotinoids, carotenes (for example alpha-carotene, beta-carotene, lycopene) and their derivatives, lipoic acid and its derivatives (for example dihydrolipoic acid), aurothioglucose, propylthiouracil and other thiols (for example thioredoxin, glutathione, cysteine, cystine, cystamine and their glycosyl, N-acetyl, methyl, ethyl, propyl, amyl, butyl and lauryl, palmitoyl, oleyl, gamma-linoleyl, cholesteryl and glyceryl esters) and their salts, dilauryl thiodipropionate, distearyl thiodipropionate, thiodipropionic acid and their derivatives (esters, ethers, peptides, lipids, nucleotides, nucleosides and salts) and sulphoximine compounds (for example buthionine sulphoximines, homocysteine sulphoximine, buthionine sulphones, pentathionine sulphoximine, hexathionine sulphoximine, heptathionine sulphoximine) in very low, acceptable doses (for example pmole to μmoles/kg), also (metal) chelating agents (for example alpha-hydroxy fatty acids, palmitic acid, phytic acid, lactoferrin), alpha-hydroxy acids (for example citric acid, lactic acid, malic acid, mandelic acid, gluconolactone, lactobionic Acid), humic acid, colic acid, colic extracts, bilirubin, biliverdin, EDTA, EGTA and their derivatives, unsaturated fatty acids and their derivatives (for example gamma-linolenic acid, linolic acid, oleic acid), folic acid and their derivatives, ubiquinone and ubiquinol and their derivatives, vitamin C and derivatives (for example ascorbyl palmitate, Mg-ascorbyl phosphate, ascorbyl acetate), tocopherols and derivatives (for example vitamin E acetate), vitamin A and derivatives (for example vitamin A palmitate) and coniferyl benzoate of benzoin resin, rutinic acid and their derivatives, butylhydroxy toluene, butylhydroxy anisole, nordihydroguaiacic acid, nordihydroguaiaretic acid, trihydroxybutyrophenone, uric acid and its derivatives, mannose and its derivatives, sesamol, sesamolin, zinc and its derivatives (for example ZnO, ZnSO₄), selenium and its derivatives (for example selenium methionine), stilbenes and their derivatives (for example stilbene oxide, trans-stilbene oxide) and suitable derivatives (salts, esters, ethers, sugars, nucleotides, nucleosides, peptides and lipids) of these said active ingredients.

The quantity of the aforementioned anti-oxidants (one or more compounds) in the preparations may be, for example, 0.0001 wt. % to 30 wt. %, 0.05 wt. % to 20 wt. %, or 1 to 10 wt. %, based on the total weight of the preparation.

Emulsions according to the invention may contain, for example the said fats, oils, waxes and other adipoids, and water and an emulsifier, as is used conventionally for such a type of formulation.

Also any mixtures of such oil and wax components can be used advantageously within the scope of the present invention. It may also optionally be advantageous to use waxes, for example cetyl palmitate, as the single lipid component of the oil phase.

Gels according to the invention may contain alcohols of low C number, for example ethanol, isopropanol, 1,2-propane diol, glycerine and water or an above-mentioned oil in the presence of a thickening agent, which for oily-alcoholic gels is preferably silicon dioxide or an aluminium silicate, for aqueous-alcoholic or alcoholic gels is preferably a polyacrylate.

Conventional highly volatile, liquefied propellants, for example hydrocarbons (propane, butane, isobutane), which may be used alone or mixed with one another, are suitable as propellants for preparations which can be sprayed from aerosol containers according to the invention. Compressed air can also advantageously be used.

Preparations of the invention may also contain filter substances that absorb UV radiation, or sunscreens, wherein the total quantity of filter substances is, for example 0.1 wt. % to 30 wt. % or 0.5 wt. % to 10 wt. %, based on the total weight of the preparation. The preparations may also serve as sunscreen agents for the skin. Such UV filter substances include, for example, the following: avenobenzene, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, and zinc oxide.

Preparations of the invention may also contain depigmenting agents, wherein the total quantity of depigmenting substances is, for example 0.1 wt. % to 30 wt. % or 0.5 wt. % to 10 wt. %, based on the total weight of the preparation. The preparations may also serve as depigmenting agents for the skin. Such substances include, for example, the following: kojic acid, methyl gentisate, hydroquinone, phytic acid, mulberry extract, grape seed extract, licorice extract, Vitamin A, Vitamin A derivates, resorcinol and its derivatives, ellagic acid, ferulic acid, azelaic acid, alpha hydroxyl acids, and Vitamin C and its derivatives.

A preparation according to the invention may include, for example, by weight of the preparation:
from about 10% to about 90% of water;
from about 0% to about 20% of at least one humectant;
from about 0% to about 20% of at least one emollient;
from about 0% to about 20% of at least one ester;
from about 0% to about 10% of at least one thickener;
from about 0% to about 2% of at least one preservative;
from about 0% to about 1% of color; and
from about 0% to about 1% of fragrance.

A preparation according to the invention may include, for example, by weight of the preparation:
from about 50% to about 90% of water;
from about 1% to about 10% of glycerin;
from about 1% to about 5% of cetyl ricinoleate;
from about 1% to about 5% of isohexadecane
from about 1% to about 5% of ceresin;
from about 0.5% to about 2.5% of sericin;
from about 0.1% to about 1% of glycosaminoglycans;
from about 0.1% to about 1% of dimethicone; and
from about 0.001% to about 30% of idebenone.

In the method according to the invention idebenone-containing compositions are applied to human, skin. The compositions may be applied once or more times per day depending on the activities the particular individual is engaged in. For example, an individual engaging in normal workday activities may wish to apply the compositions twice a day, once in the morning, and once in the evening, in conjunction with normal grooming. On the other hand, if the individual plans outdoor activities such as sunbathing and athletics, the compositions may be applied prior to, and during, such activities, much like a sunscreen composition is applied periodically during the day. The compositions may be used for hyperpigmentation on the face and neck, by applying appropriate idebenone compositions to the face and neck areas. However, the idebenone compositions may also be applied to the entire body, particularly areas which are not covered by clothing, such as the arms, neck, and lower legs.

In an embodiment of the present invention, a topical preparation directed to reducing an occurrence of hyperpigmentation in human skin is made available, for example for sale to wholesale or retail buyers by a wholesale or retail entity. The preparation may be made available as a product advertised or marketed to reduce hyperpigmentation. Also provided is information on applying the topical preparation to the skin, for example directions for use and/or indications on the product package. The topical preparation includes an amount of an agent effective to reduce an occurrence of hyperpigmentation in human skin. The agent includes idebenone or a derivative of idebenone.

### EXAMPLE 1

A 0.5% idebenone lotion Formula 1 was prepared as follows:

Formula 1:

| | W/W |
|---|---|
| Ingredient (INCI Name) | % |
| Water Soluble Ingredients: | |
| Purified Water (Aqua) | 53.83 |
| Glycerin | 10.00 |
| Sodium PCA | 3.00 |
| Potato Starch Modified | 2.00 |
| Xanthan Gum | 0.28 |
| Methylparaben | 0.25 |
| Water (and) Titanium Dioxide (C177891) (and) Propylene | 2.75 |
| Glycol (and) Styrene Acrylates Copolymer (and) | |
| Hydrolyzed Corn Starch (and) Ammonium Hydroxide | |
| (and) Phenoxyethanol (and) Methylparaben (and) | |
| Ethylparaben (and) Butylparaben (and) Propylparaben | |
| Lipid Soluble Ingredients: | |
| Isohexadecane | 2.75 |
| Allantoin Glycyrrhetinic Acid | 0.10 |
| Glyceryl Stearate (and) PEG-100 Stearate | 1.60 |
| C30-45 Alkyl Methicone | 0.50 |
| Stearyl Alcohol (and) Ceteareth-20 | 2.20 |
| Salicyloyl Phytosphingosine | 0.10 |
| Sucrose Cocoate | 4.29 |
| BHT | 0.15 |
| Propylparaben | 0.15 |
| Dimethicone (and) Polysilicone-11 | 4.00 |
| Glycol Distearate | 1.50 |
| Heat Sensitive Ingredients: | |
| Sericin | 1.50 |
| Glycosaminoglycans | 0.50 |
| Acetyl Hexapeptide-3 | 4.00 |
| Isopropyl Lauroyl Sarcosinate | 2.00 |
| Hydroxydecyl Ubiquinone (Idebenone) | 0.50 |
| Bisabolol | 0.50 |
| Aluminum Starch Octenylsuccinate (and) Lauroyl Lysine | 1.30 |
| Diazolidinyl Urea (and) Iodopropynyl Butylcarbamate | 0.30 |

To prepare Formula 1, ingredients were separated into groups based on water soluble, lipid soluble, or heat sensitive and added to the formula in the following manner:

Step 1 (Water Soluble Ingredients): To a suitable size container equipped with high speed agitation and heating and cooling capacities, water was added and heated to 75° C. When the temperature reached approximately 75° C., heating was discontinued and temperature maintained at 75° C. Water soluble thickeners, emulsifiers, preservatives, and colorants were individually added to the batch with moderate agitation. Mixing was continued until the water phase was smooth, homogenous and uniform. Batch temperature was maintained at 75° C.

Step 2 (Oil Soluble Ingredients): In a separate container of suitable size equipped with heating and mixing capacities all oil soluble ingredients were combined and heated to 75-80° C. with adequate agitation until all solids were melted and the phase was uniform. Temperature was maintained at 75° C.

Step 3 (Emulsification): When the oil phase was both uniform and at 75° C., it was added to the water phase with continuous, rapid mixing until the mixture was uniform, at which point agitation was decreased and cooling of the mixture initiated.

Step 4 (Heat Sensitive Ingredients): Once the temperature was decreased, the remaining ingredients were added between 40° C. to 60° C. with adequate agitation between each addition. Cooling was continued with slow agitation until the mixture reached 30° C. at which time the mixture was considered complete.

### EXAMPLE 2

The idebenone-containing Formula 1 of Example 1 was applied to human skin in a six-week hyperpigmentation study.

This study used 10 female subjects between the ages of 18 and 65 years who possess moderate skin hyperpigmentation. At the Baseline visit (week 0), all subjects completed an informed consent and a photography consent. Following the consent process, the investigator performed a medical history and examined the appearance of the face to determine the subject's appropriateness for inclusion in the study. Subjects were asked to discontinue their own personally selected hyperpigmentation skin care product(s) and replace them with the test product containing idebenone. A variety of evaluations were performed at baseline, week 3, and week 6. The investigator assessed the subject's facial appearance by rating the subject's level of hyperpigmentation on a 5-point ordinal scale (0=no change; 1=slight; 2=moderate; 3=Marked; and 4=Severe). Additionally, 35 mm photography was performed. The treatment period was 6 weeks with the final study visit occurring at week 6. Subjects returned all unused test products to the study coordinator at this visit. Subjects, who in the investigator's opinion, appear to be experiencing problems due to the treatment were discontinued at the investigator's discretion at any time during the study. An unscheduled visit may be performed for this purpose.

This study was comprised of female subjects presenting with the clinical appearance of moderate hyperpigmentation. Ten female subjects who meet the inclusion and exclusion criteria, noted below, were enrolled.

The following items represent the inclusion criteria:
1. Subjects must be diagnosed by the investigator with moderate hyperpigmentation.
2. Subjects must be female and between 18 and 65 years of age with no known medical conditions that, in the investigator's opinion, may interfere with study participation.
3. Subjects must discontinue all current hyperpigmentation skin care products.
4. Subjects must provide written informed consent and photography consent.

The following represent the exclusion criteria:
1. Any dermatological disorder or personal appearance issue, which in the investigator's opinion, may interfere with the accurate evaluation of the subject's face.
2. Subjects who have demonstrated a previous hypersensitivity reaction to any ingredients in the study products.
3. Concurrent therapy with any medication either topical or oral that might interfere with the study.
4. Subjects who have undergone any surgical treatment to the tissues of the face.
5. Subjects who are not willing to discontinue all prescription or OTC cosmeceutical preparations to the face.
6. Subjects who have participated in another clinical trial or have taken an experimental drug within the past 30 days.
7. Subjects who are pregnant, breast-feeding, or planning a pregnancy.
8. Subjects who are unwilling or unable to comply with the requirements of the protocol.

The use of the following medications and skin care products were prohibited while enrolled in the study:
1. Any topical cosmeceutical to the face.
2. Any prescription topical medication to the face, such as topical corticosteroids or retinoids, hydroquinones during the study period.

If the subjects are actively using a prescription or OTC treatment product for hyperpigmentation, a one-week washout period was required.

Results: After 6 weeks of product use, a mean percent reduction of 50% in hyperpigmentation was observed.

The results are summarized in Tables I below.

TABLE I
Percent Improvement in Hyperpigmentation

| | Pre- | Post- |
|---|---|---|
| Subject | Value | Value |
| 1 | 2 | 1 |
| 2 | 1 | 1 |
| 3 | 3 | 1 |
| 4 | 2 | 1 |
| 5 | 2 | 1 |
| 6 | 2 | 1 |
| 7 | 2 | 1 |
| 8 | 2 | 1 |
| 9 | 2 | 1 |
| 10 | 2 | 1 |
| Mean | 2 | 1 |
| % Change from Baseline | | 50 |

Conclusion: Based on the data obtained from this study, the application of a 0.5% idebenone lotion provided significant benefits in the reduction of hyperpigmentation in photodamaged skin as evidence by the changes observed.

### EXAMPLE 3

A 1.0% idebenone lotion Formula 2 was prepared as follows:

Formula 2:

| | W/W |
|---|---|
| Ingredient (INCI Name) | % |
| Water Soluble Ingredients: | |
| Purified Water (Aqua) | 53.08 |
| Glycerin | 10.00 |
| Sodium PCA | 3.00 |
| Potato Starch Modified | 2.00 |
| Xanthan Gum | 0.28 |
| Methylparaben | 0.25 |
| Water (and) Titanium Dioxide (C177891) (and) Propylene | 2.75 |
| Glycol (and) Styrene Acrylates Copolymer (and) | |
| Hydrolyzed Corn Starch (and) Ammonium Hydroxide | |
| (and) Phenoxyethanol (and) Methylparaben (and) | |
| Ethylparaben (and) Butylparaben (and) Propylparaben | |
| Lipid Soluble Ingredients: | |
| Isohexadecane | 2.75 |
| Allantoin Glycyrrhetinic Acid | 0.10 |
| Glyceryl Stearate (and) PEG-100 Stearate | 1.60 |
| C30-45 Alkyl Methicone | 0.50 |
| Stearyl Alcohol (and) Ceteareth-20 | 2.20 |
| Salicyloyl Phytosphingosine | 0.10 |
| Sucrose Cocoate | 4.29 |
| BHT | 0.15 |
| Propylparaben | 0.15 |
| Dimethicone (and) Polysilicone-11 | 4.00 |
| Glycol Distearate | 1.50 |
| Heat Sensitive Ingredients: | |
| Sericin | 1.50 |
| Glycosaminoglycans | 0.50 |
| Acetyl Hexapeptide-3 | 4.00 |
| Isopropyl Lauroyl Sarcosinate | 2.25 |
| Hydroxydecyl Ubiquinone (Idebenone) | 1.00 |
| Bisabolol | 0.50 |
| Aluminum Starch Octenylsuccinate (and) Lauroyl Lysine | 1.30 |
| Diazolidinyl Urea (and) Iodopropynyl Butylcarbamate | 0.30 |

To prepare Formula 2, ingredients were separated into groups based on water soluble, lipid soluble, or heat sensitive and added to the formula in steps as described in Example 1 above.

### EXAMPLE 4

The idebenone-containing Formula 2 of Example 3 was applied to human skin in a six-week hyperpigmentation study performed in the manner of the six-week study described in Example 2 above.

Results: After 6 weeks of product use, a mean percent reduction of 50% in hyperpigmentation was observed.

The results are summarized in Table II below.

TABLE II
Percent Improvement in Hyperpigmentation

| | Pre- | Post- |
|---|---|---|
| Subject | Value | Value |
| 1 | 3 | 2 |
| 2 | 2 | 1 |
| 3 | 2 | 1 |
| 4 | 2 | 1 |
| 5 | 4 | 1 |
| 6 | 2 | 1 |
| 7 | 2 | 1 |
| 8 | 1 | 0.5 |
| 9 | 1 | 1 |
| 10 | 2 | 1 |
| Mean | 2.1 | 1.05 |
| % Change from Baseline | | 50 |

Conclusion: Based on the data obtained from this study, the 1.0% idebenone lotion provided significant benefits in the reduction of hyperpigmentation in photodamaged skin via the changes observed.

While the invention has been described in connection with individual embodiments, it is not intended to limit the scope of the invention to the particular form set forth, but, on the contrary, it is intended to cover such alternatives, modifications and equivalents as may be included within the spirit and scope of the invention as defined by the appended claims.

1. A method for reducing an occurrence of hyperpigmentation in human skin, comprising applying a topical preparation to the skin, the preparation comprising an amount of an agent effective to reduce an occurrence of hyperpigmentation in human skin, the agent comprising idebenone or a derivative of idebenone.

2. The method as recited in claim 1 wherein the applying is performed after the hyperpigmentation has occurred.

3. The method as recited in claim 1 wherein the idebenone or derivative of idebenone has a concentration of from about 0.001% to about 30% by weight of the topical preparation.

4. The method as recited in claim 1 wherein the idebenone or derivative of idebenone has a concentration of from about 0.01% to about 10.0% by weight of the topical preparation.

5. The method as recited in claim 1 wherein the idebenone or derivative of idebenone has a concentration of from about 0.10% to about 2.0% by weight of the topical preparation.

6. The method as recited in claim 1 wherein the idebenone or derivative of idebenone has a concentration of from about 0.5% to about 1.0% by weight of the topical preparation.

7. The method as recited in claim 1 wherein the derivative of idebenone is selected from the group consisting of a salt of idebenone, an ester of idebenone, and a protein-bound form of idebenone.

8. The method as recited in claim 1 wherein the topical preparation is in the form of at least one of a lotion, a cream, a gel, a solution, a spray, a cleanser, a powder, an ointment, a wax, a lipstick, a soap, a shampoo, a hydroalcoholic solution, a suspension, a scrub, a saturated pad, and a skin or hair conditioning agent.

9. The method as recited in claim 1 wherein the applying is performed prior to an event known or suspected to cause hyperpigmentation.

10. The method as recited in claim 9 wherein the event is at least one of an exposure to ultraviolet radiation, an exposure to a chemical, an exposure to a cosmetic and an exposure to a drug.

11. The method as recited in claim 1 wherein the applying is performed at least once a day.

12. The method as recited in claim 1 wherein the applying is performed prior to engaging in outdoor activities.

13. The method as recited in claim 1 wherein the skin is at least one of facial skin, skin on the neck, skin on the arms, skin on the hands and skin on the legs.

14. The method as recited in claim 1 wherein the topical preparation is at least one of a cosmetic preparation, a dermatological preparation, an over-the-counter preparation, and a pharmaceutical preparation.

15. The method as recited in claim 0.1 wherein the topical preparation further comprises at least one antioxidant.

16. The method as recited in claim 1 wherein the topical preparation further comprises at least one antioxidant selected from the group consisting of Vitamin E, Vitamin C, Glutathione, Superoxide Dismutase, Catalase, Co-Enzyme Q10, a botanical extract, alpha lipoic acid, N-furfuryladenine and an antioxidant polyphenol.

17. The method as recited in claim 1 wherein the topical preparation further comprises at least one ultraviolet filter substance.

18. The method as recited in claim 1 wherein the topical preparation further comprises at least one ultraviolet filter substance selected from the group consisting of avenobenzene, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, and zinc oxide.

19. The method as recited in claim 1 wherein the topical preparation further comprises at least one depigmenting substance.

20. The method as recited in claim 1 wherein the topical preparation further comprises at least one depigmenting substance selected from the group consisting of kojic acid, methyl gentisate, hydroquinone, phytic acid, mulberry extract, grape seed extract, licorice extract, Vitamin A, a Vitamin A derivate, resorcinol or a derivative thereof, ellagic acid, ferulic acid, azelaic acid, an alpha hydroxyl acid and Vitamin C or derivative thereof.

21. A method for reducing an occurrence of hyperpigmentation in human skin, comprising:
providing a person in need of a reduction of an occurrence of hyperpigmentation, the occurrence of hyperpigmentation being responsive to an application of idebenone or a derivative of idebenone; and
applying a topical preparation to the skin, the preparation comprising an amount of an agent effective to reduce the occurrence of hyperpigmentation in human skin, the agent comprising idebenone or a derivative of idebenone.

22. The method as recited in claim 21 wherein the applying is performed after the hyperpigmentation has occurred.

23. The method as recited in claim 21 wherein the idebenone or derivative of idebenone has a concentration of from about 0.001% to about 30% by weight of the topical preparation.

24. The method as recited in claim 21 wherein the derivative of idebenone is selected from the group consisting of a salt of idebenone, an ester of idebenone, and a protein-bound form of idebenone.

25. The method as recited in claim 21 wherein the topical preparation is in the form of at least one of a lotion, a cream, a gel, a solution, a spray, a cleanser, a powder, an ointment, a wax, a lipstick, a soap, a shampoo, a hydroalcoholic solution, a suspension, a scrub, a saturated pad, and a skin or hair conditioning agent.

26. The method as recited in claim 21 wherein the topical preparation further comprises at least one ultraviolet filter substance.

27. The method as recited in claim 21 wherein the topical preparation further comprises at least one ultraviolet filter substance selected from the group consisting of avenobenzene, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, and zinc oxide.

28. The method as recited in claim 21 wherein the topical preparation further comprises at least one of an antioxidant and a depigmenting substance.

29. A method for reducing occurrence of hyperpigmentation in human skin, the method comprising:
making available a topical preparation directed to reducing an occurrence of hyperpigmentation in human skin, the topical preparation comprising an amount of an agent effective to reduce the occurrence of hyperpigmentation in human skin, the agent comprising idebenone or a derivative of idebenone; and
providing an information on applying the topical preparation to the skin.

30. The method as recited in claim 29 wherein the information includes an indication that the applying is performed for the purpose of reducing the occurrence of hyperpigmentation in human skin.

31. The method as recited in claim 29 wherein the making available includes engaging in marketing, advertising or indicating that the topical preparation is for reducing an occurrence of hyperpigmentation in human skin.

32. The method as recited in claim 29 wherein the applying is performed after the hyperpigmentation has occurred.

33. The method as topical preparation recited in claim 29 wherein the idebenone or derivative of idebenone has a concentration of from about 0.001% to about 30% by weight of the topical preparation.

34. The method as recited in claim 29 wherein the derivative of idebenone is selected from the group consisting of a salt of idebenone, an ester of idebenone, and a protein-bound form of idebenone.

35. The method as recited in claim 29 wherein the topical preparation is in the form of at least one of a lotion, a cream, a gel, a solution, a spray, a cleanser, a powder, an ointment, a wax, a lipstick, a soap, a shampoo, a hydroalcoholic solution, a suspension, a scrub, a saturated pad, and a skin or hair conditioning agent.

36. The method as recited in claim 29 wherein the topical preparation further comprises at least one ultraviolet filter substance.

37. The method as recited in claim 29 wherein the topical preparation further comprises at least one ultraviolet filter substance selected from the group consisting of avenobenzene, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, and zinc oxide.

38. A topical preparation for reducing an occurrence of hyperpigmentation in human skin, comprising:
at least one of an ultraviolet filter substance, a depigmentation substance and an antioxidant; and
an amount of idebenone or a derivative of idebenone effective to reduce an occurrence of hyperpigmentation in human skin.

39. The preparation as recited in claim 38 wherein the ultraviolet filter substance is selected from the group consisting of avenobenzene, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, and zinc oxide.

40. The preparation as recited in claim 38 wherein the antioxidant is selected from the group consisting of Vitamin E, Vitamin C, Glutathione, Superoxide Dismutase, Catalase, Co-Enzyme Q10, a botanical extract, alpha lipoic acid, N-furfuryladenine and an antioxidant polyphenol.

41. The preparation as recited in claim 38 wherein the depigmenting substance is selected from the group consisting of kojic acid, methyl gentisate, hydroquinone, phytic acid, mulberry extract, grape seed extract, licorice extract, Vitamin A, a Vitamin A derivate, resorcinol or a derivative thereof, ellagic acid, ferulic acid, azelaic acid, an alpha hydroxyl acid and Vitamin C or derivative thereof.

42. The preparation as recited in claim 38 wherein the hyperpigmentation occurs upon an exposure of the skin to at least one of ultraviolet radiation, a chemical, a cosmetic and a drug.

43. The preparation as recited in claim 38 wherein the idebenone or derivative of idebenone has a concentration of from about 0.001% to about 30% by weight of the preparation.

44. The preparation as recited in claim 38 wherein the derivative of idebenone is selected from the group consisting of a salt of idebenone, an ester of idebenone, and a protein-bound form of idebenone.

45. The preparation as recited in claim 38 wherein the preparation is in the form of at least one of a lotion, a cream, a gel, a solution, a spray, a cleanser, a powder, an ointment, a wax, a lipstick, a soap, a shampoo, and a hydroalcoholic solution.
